(19) United States
(12) Patent Application Publication
(10) Pub. No.: US 2025/0171755 A1
(43) Pub. Date: May 29, 2025

(54) VARIANT ALICYCLOBACILLUS CAS12b PROTEINS WITH IMPROVED DNA CLEAVAGE SELECTIVITY AND METHODS OF USE

(71) Applicant: The Board of Regents of the University of Oklahoma, Norman, OK (US)

(72) Inventor: Rakhi Rajan, Norman, OK (US)

(21) Appl. No.: 18/929,841

(22) Filed: Oct. 29, 2024

Related U.S. Application Data
(63) Continuation of application No. 16/946,689, filed on Jul. 1, 2020, now Pat. No. 11,459,552.

(51) Int. Cl. C12N 9/22; C12N 15/113; C12N 15/74

(52) U.S. Cl. CPC C12N 9/22; C12N 15/113; C12N 15/74; C12N 2310/20; C12N 2800/80

(57) ABSTRACT

Bridge helix-modified variant Cas12a and Cas12b proteins having improved DNA cleavage selectivity in comparison to wild type versions of the Cas12a and Cas12b proteins, nucleic acids encoding the variant proteins, host cells containing the nucleic acids, and methods of their use. In particular embodiments, the at least one substitution or at least two substitutions are to proline. In certain embodiments, bridge helix-modified proteins are variants of the Cas12a proteins from Francisella tularensis novicida U112, Lachnospiraceae bacterium, and Acidaminococcus sp, and of the Cas12b protein from Alicyclobacillus acidoterrestris.

### CROSS REFERENCE TO RELATED APPLICATIONS

The present patent application is a continuation of U.S. Ser. No. 17/704,317, filed Mar. 25, 2022, which is a continuation of U.S. Ser. No. 16/946,689, filed Jul. 1, 2020, which is a continuation-in-part of U.S. Ser. No. 16/570,555, filed Sep. 13, 2019, which claims priority to U.S. Provisional Patent Application Ser. Nos. 62/730,890, filed on Sep. 13, 2018, and 62/870,472, filed on Jul. 3, 2019, the entireties of which are hereby expressly incorporated herein by reference.

### GOVERNMENT SUPPORT

This invention was made with government support under National Science Foundation Grant MCB-1716423 and National Institutes of Health Grant P20GM103640. The government has certain rights in the invention.

### SEQUENCE LISTING STATEMENT

The ASCII filed, entitled “820233.02563.xml”, created on Oct. 29, 2024, comprising 10,222 bytes, submitted concurrently with the filing of this application is incorporated herein by reference.

### BACKGROUND

CRISPR-Cas (clustered regularly interspaced short palindromic repeats-CRISPR associated) systems are RNA-guided nucleic acid targeting machinery in bacteria and archaca that provide adaptive immunity against intruding genomic materials such as phages. These systems have been repurposed into powerful gene editing tools over the past decade. Cas9 and Cas12a are two proteins from the CRISPR systems that are currently available for gene editing. These multidomain proteins can cleave DNA by recognizing a protospacer-adjacent motif (PAM) region and specifically using an RNA as a guide. In the case of the Cas12a protein, crRNA (CRISPR-RNA) is used as a guide. The RNA-DNA complementarity is essential for sequence-specific target DNA cleavage by Cas proteins. Within these proteins is a long arginine rich helix called bridge helix (BH) that is indispensable for their activity. The BH connects the nuclease (NUC) and recognition (REC) lobes. The NUC lobe is significant as it contains the domain responsible for PAM recognition and the endonuclease domain, RuvC. We recently showed that the BH region in Cas9 influences selectivity in target DNA cleavage. It is of interest to identify the role of the BH region for DNA cleavage specificity by Cas12 proteins. It is to that end that the present disclosure is directed.

### BRIEF DESCRIPTION OF THE DRAWINGS

FIG. 1 shows the amino acid sequence of Francisella tularensis novicida strain U112 Cas12a (FnCas12a) protein (SEQ ID NO:1). The bridge helix (BH) sequence is represented in boldface.

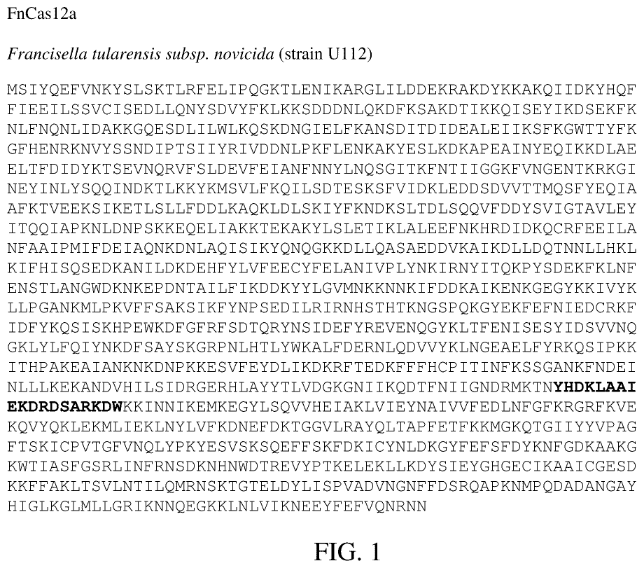

FIG. 2 shows the amino acid sequence of Lachnospiraceae bacterium strain ND2006 Cas12a (LbCas12a) protein (SEQ ID NO:2). The bridge helix (BH) sequence is represented in boldface.

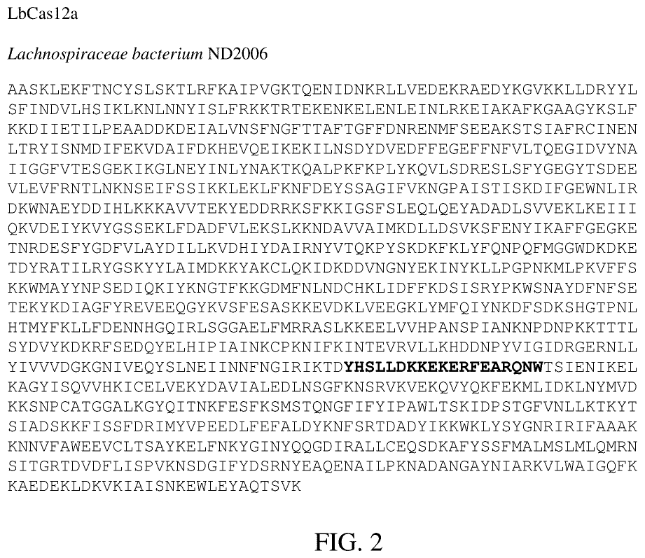

FIG. 3 shows the amino acid sequence of Acidaminococcus sp strain BV3L6 Cas12a (AsCas12a) protein (SEQ ID NO:3). The bridge helix (BH) sequence is represented in boldface.

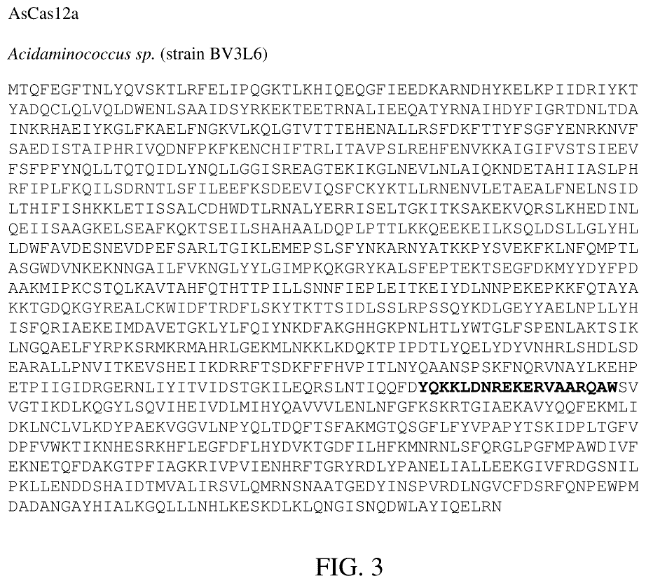

FIG. 4 shows the amino acid sequence of Alicyclobacillus acidoterrestris strain 49025 Cas12b (AaCas12b) protein (SEQ ID NO:4). The bridge helix (BH) sequence is represented in boldface.

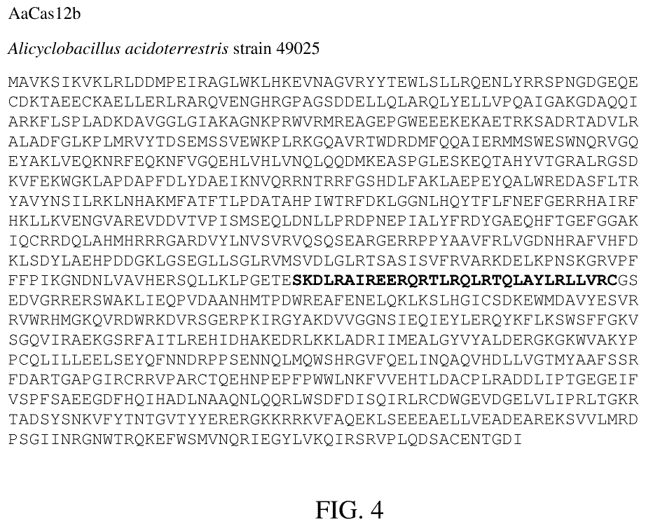

### DETAILED DESCRIPTION

The present disclosure is directed to variant Cas12a and Cas12b proteins comprising at least one amino acid substitution in the bridge helix (BH) portion of the protein, and which have improved DNA cleavage selectivity in comparison to the wild type version of the Cas12a or Cas12b protein, respectively. Certain embodiments comprise at least two amino acid substitutions in the BH portion. In particular embodiments, the at least one substitution or at least two substitutions are to proline. In certain embodiments, the present disclosure is also directed to BH variants of Cas12a from Francisella tularensis novicida U112, Lachnospiraceae bacterium, and Acidaminococcus sp, and of Cas12b from Alicyclobacillus acidoterrestris.

More particularly, certain non-limiting embodiments include, in Cas12a from Francisella tularensis novicida U112, substitutions in any one or more of positions 956, 957, 961, 962, 969, and 970 of the amino acid sequence SEQ ID NO:1. The at least one amino acid substitution in the modified BH region may be selected from the group of amino acids consisting of ala, arg, asn, asp, cys, glu, gln, gly, his, ile, leu, lys, met, phe, pro, ser, thr, trp, tyr, and val. In non-limiting embodiments, the wild type amino acid may be substituted with proline, e.g., Lysine 956 and/or Leucine 957 mutated to Proline 956 and/or Proline 957, respectively; Glutamic acid 961 and/or Lysine 962 mutated to Proline 961 and/or Proline 962, respectively; and Lysine 969 and/or Aspartic acid 970 mutated to Proline 969 and/or Proline 970, respectively.

Other embodiments include, in Cas12a from Lachnospiraceae bacterium, substitutions in any one or more of positions 875, 876, 880, 881, 888, and 889 of the amino acid sequence SEQ ID NO: 2. The at least one amino acid substitution in the modified BH region may be selected from the group of amino acids consisting of ala, arg, asn, asp, cys, glu, gln, gly, his, ile, leu, lys, met, phe, pro, ser, thr, trp, tyr, and val. In non-limiting embodiments, the wild type amino acid may be substituted with proline, e.g., Leu 875 and/or Leu 876 mutated to Proline 875 and/or Proline 876, respectively; Glu 880 and/or Lys 881 mutated to Proline 880 and/or Proline 881, respectively; and Gln 888 and/or Asn 889 mutated to Proline 888 and/or Proline 889, respectively.

Other embodiments include, in Cas12a from Acidaminococcus sp, substitutions in any one or more of positions 943, 944, 948, 949, 956, and 957, of the amino acid sequence SEQ ID NO: 3. The at least one amino acid substitution in the modified BH region may be selected from the group of amino acids consisting of ala, arg, asn, asp, cys, glu, gln, gly, his, ile, leu, lys, met, phe, pro, ser, thr, trp, tyr, and val. In non-limiting embodiments, the wild type amino acid may be substituted with proline, e.g., Lys 943 and/or Leu 944 mutated to Proline 943 and/or Proline 944, respectively; Glu 948 and/or Lys 949 mutated to Proline 948 and/or Proline 949, respectively; and Gln 956 and/or Ala 957 mutated to Proline 956 and/or Proline 957, respectively.

Other embodiments include, in Cas12b from Alicyclobacillus acidoterrestris, substitutions in any one or more of positions 628-658 of the amino acid sequence SEQ ID NO:4. The at least one amino acid substitution in the modified BH region may be selected from the group of amino acids consisting of ala, arg, asn, asp, cys, glu, gln, gly, his, ile, leu, lys, met, phe, pro, ser, thr, trp, tyr, and val. In non-limiting embodiments, the wild type amino acid may be substituted with proline, e.g., Leu 631 and/or Arg 632 mutated to Pro 631 and/or Pro 632, respectively; Gln 644 and/or Leu 645 mutated to Pro 644 and/or Pro 645, respectively; Leu 654 and/or Leu 655 mutated to Pro 654 and/or Pro 655, respectively; and/or Leu 655 and/or Val 656 mutated to Pro 655 and/or Pro 656, respectively.

Before further description of various embodiments of the present disclosure in more detail by way of exemplary description, examples, and results, it is to be understood that the present disclosure is not limited in application to the details of methods, constructs, cells, and compositions as set forth in the following description. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that other embodiments of the inventive concepts may be practiced without these specific details. In other instances, features which are well known to persons of ordinary skill in the art have not been described in detail to avoid unnecessary complication of the description.

All patents, published patent applications, and non-patent publications referenced in any portion of this application, including U.S. Ser. No. 16/570,555, filed Sep. 13, 2019, and U.S. Provisional Patent Application Ser. Nos. 62/730,890, filed on Sep. 13, 2018, and 62/870,472, filed on Jul. 3, 2019, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art.

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z. Further, unless otherwise required by context, singular terms shall include pluralities and plural terms shall include the singular. Where used herein, the specific term “single” is limited to only “one”. Use of the word “we” as a pronoun herein refers generally to laboratory personnel or other contributors who assisted in laboratory procedures and data collection and is not intended to represent an inventorship role by said laboratory personnel or other contributors in any subject matter disclosed herein.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the constructs, cells, compositions and methods used, or the variation that exists among the study objects. Further, in this detailed description and the appended claims, each numerical value (e.g., temperature or time) should be read once as modified by the term “about” (unless already expressly so modified), and then read again as not so modified unless otherwise indicated in context. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

Also, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1). Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 1-20, 10-50, 50-100, 100-1,000, 1,000-3,000, 2,000-4,000, etc.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

Protein variants disclosed herein may comprise conservative substitutions in portions of the BH region, as well as in other regions and domains of the protein. Substitutions may be selected from the natural amino acids. The natural amino acids include and may be referred to herein by the following designations: alanine: ala or A; arginine: arg or R; asparagine: asn or N; aspartic acid: asp or D; cysteine: cys or C; glutamic acid: glu or E; glutamine: gln or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V. Amino acids may be D or L enantiomers.

For purposes of classifying amino acids substitutions as conservative or nonconservative, amino acids are grouped in one embodiment as follows: Group I (hydrophobic side chains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Conservative substitutions involve substitutions between amino acids in the same group. Non-conservative substitutions constitute exchanging a member of one of these groups for a member of another.

Tables of exemplary conservative amino acid substitutions have been constructed and are known in the art. In certain embodiments herein which reference possible substitutions, examples of interchangeable amino acids include, but are not limited to the following: arginine and lysine; glutamate and aspartate; serine and threonine; glutamine and asparagine; and valine, leucine and isoleucine. In other embodiments, the following substitutions can be made: Ala (A) by leu, ile, or val; Arg (R) by gln, asn, or lys; Asn (N) by his, asp, lys, arg, or gln; Asp (D) by asn, or glu; Cys (C) by ala, or ser; Gln (Q) by glu, or asn; Glu (E) by gln, or asp; Gly (G) by ala; His (H) by asn, gln, lys, or arg; Ile (I) by val, met, ala, phe, or leu; Leu (L) by val, met, ala, phe, or ile; Lys (K) by gln, asn, or arg; Met (M) by phe, ile, or leu; Phe (F) by leu, val, ile, ala, or tyr; Pro (P) by ala; Ser(S) by thr; Thr (T) by ser; Trp (W) by phe, or tyr; Tyr (Y) by trp, phe, thr, or ser; and Val (V) by ile, leu, met, phe, or ala.

Other considerations for amino acid substitutions include whether or not the residue is located in the interior of a protein or is solvent—(i.e., externally) exposed. For interior residues, conservative substitutions include for example: Asp and Asn; Ser and Thr; Ser and Ala; Thr and Ala; Ala and Gly; Ile and Val; Val and Leu; Leu and Ile; Leu and Met; Phe and Tyr; and Tyr and Trp. For solvent-exposed residues, conservative substitutions include for example: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; and Phe and Tyr.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally-occurring purine or pyrimidine base found in DNA (e.g., an adenine “A,” a guanine “G,” a thymine “T” or a cytosine “C”) or RNA (e.g., an “A,” a “G,” a uracil “U” or a “C”). The term nucleobase also includes non-natural bases as described below. The term “nucleic acid” encompasses the terms “oligonucleotide” and “polynucleotide,” each as a subgenus of the term “nucleic acid.” In certain embodiments, the present disclosure is directed to nucleic acids (DNA and RNA) which encode the variant Cas9 proteins described.

As used herein, the terms “complementary” or “complement” also refer to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

In at least certain embodiments, the Cas12 variants described herein have at least 90% identity, or at least 91% identity, or at least 92% identity, or at least 93% identity, or at least 94% identity, or at least 95% identity, or at least 96% identity, or at least 97% identity, or at least 98% identity, or at least 99% identity to the corresponding wild type versions of the Cas12 proteins.

The term “homologous” or “% identity” as used herein means a nucleic acid (or fragment thereof), or a protein (or a fragment thereof) having a degree of homology to the corresponding natural reference nucleic acid, or protein, that is at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% identical thereto. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

Percentage sequence identities can be determined with protein sequences maximally aligned by the Kabat numbering convention. After alignment, if a particular polypeptide region is being compared with the same region of a reference polypeptide, the percentage sequence identity between the subject and reference polypeptide region is the number of positions occupied by the same amino acid in both the subject and reference polypeptide region divided by the total number of aligned positions of the two regions, with gaps not counted, multiplied by 100 to convert to percentage.

In one embodiment “% identity” represents the number of amino acids which are identical at corresponding positions in two sequences of a protein having the same or similar activity. For example, two amino acid sequences each having 100 residues will have at least 90% identity when 90 of the amino acids at corresponding positions are the same. Similarly, in one embodiment “% identity” represents the number of nucleotides which are identical at corresponding positions in two sequences of a nucleic acid encoding the same or similar polypeptides. For example, two nucleic acid sequences each having 100 nucleotides will have 90% identity when 90 of the nucleotides in homologous positions are the same.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3:266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

As used herein, “hybridization,” “hybridizes” or “capable of hybridizing” is understood to mean the forming of a double or triple stranded molecule or a molecule with partial double or triple stranded nature. The term “anneal” as used herein is synonymous with “hybridize.” The term “hybridization,” “hybridize(s)” or “capable of hybridizing” encompasses the terms “stringent condition(s)” or “high stringency” and the terms “low stringency” or “low stringency condition(s).”

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Non-limiting applications include isolating a nucleic acid, such as a gene or a nucleic acid segment thereof, or detecting at least one specific mRNA transcript or a nucleic acid segment thereof, and the like. Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid, the length and nucleobase content of the target sequence, the charge composition of the nucleic acid, and to the presence or concentration of formamide, tetramethylammonium chloride or other solvent in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned varying conditions of hybridization to achieve varying degrees of selectivity of a nucleic acid towards a target sequence are used. In a non-limiting example, identification or isolation of a related target nucleic acid that does not hybridize to a nucleic acid under stringent conditions may be achieved by hybridization at low temperature and/or high ionic strength. Such conditions are termed “low stringency” or “low stringency conditions,” and non-limiting examples of low stringency include hybridization performed at about 0.15 M to about 0.9 M NaCl at a temperature range of about 20° C. to about 50° C. Of course, it is within the skill of one in the art to further modify the low or high stringency conditions to suit a particular application.

In certain embodiments herein, a “gene” refers to a nucleic acid that is transcribed. In certain aspects, the gene includes regulatory sequences involved in transcription, or message production or composition. In particular embodiments, the gene comprises transcribed sequences that encode for a protein, polypeptide or peptide. As will be understood by those in the art, this function term “gene” includes both genomic sequences, RNA or cDNA sequences or smaller engineered nucleic acid segments, including nucleic acid segments of a non-transcribed part of a gene, including but not limited to the non-transcribed promoter or enhancer regions of a gene. Smaller engineered gene nucleic acid segments may express, or may be adapted to express using nucleic acid manipulation technology, proteins, polypeptides, domains, peptides, fusion proteins, mutants and/or the like.

The term encoding” as used herein refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence” or “nucleic acid” encoding an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “bind,” “binds,” or “interacts with” means that one molecule recognizes and adheres to a particular second molecule in a sample or organism, but does not substantially recognize or adhere to other structurally unrelated molecules in the sample.

By the term “specifically binds,” as used herein with respect to an antibody, is meant an antibody which recognizes a specific antigen, but does not substantially recognize or bind other molecules in a sample.

A “host cell,” as used herein, denotes an in vivo or in vitro eukaryotic cell, a prokaryotic cell, or a cell from a multicellular organism (e.g., a cell line) cultured as a unicellular entity, which eukaryotic or prokaryotic cells can be, or have been, used as recipients for a nucleic acid (e.g., an expression vector that comprises a nucleotide sequence of interest), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject prokaryotic host cell is a genetically modified prokaryotic host cell (e.g., a bacterium), by virtue of introduction into a suitable prokaryotic host cell of a heterologous nucleic acid, e.g., an exogenous nucleic acid that is foreign to (not normally found in nature in) the prokaryotic host cell, or a recombinant nucleic acid that is not normally found in the prokaryotic host cell; and a subject eukaryotic host cell is a genetically modified eukaryotic host cell, by virtue of introduction into a suitable eukaryotic host cell of a heterologous nucleic acid, e.g., an exogenous nucleic acid that is foreign to the eukaryotic host cell, or a recombinant nucleic acid that is not normally found in the eukaryotic host cell.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which includes an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, et al. Examples of viral vectors include, but are not limited to, adenoviral vectors, adeno-associated virus vectors, and retroviral vectors. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by attenuating the HIV virulence genes, for example, the genes env, vif, vpr, vpu, and nef are deleted making the vector biologically safe. In other embodiments of the present disclosure, a gamma retrovirus may be used as the transfecting agent.

Where used herein the term “wild-type” refers to the typical form (genotype and/or phenotype) of a bacterium, gene, nucleic acid, protein, or peptide as it occurs in nature and/or is the most common form in a natural population. In reference to a gene or nucleic acid, the term “mutation” refers to a gene or nucleic acid comprising an alteration in the wild type, such as but not limited to, a nucleotide deletion, insertion, and/or substitution. A mutation in a gene or nucleic acid generally results in either inactivation, decrease in expression or activity, increase in expression or activity, or another altered property of the gene or nucleic acid. In reference to a protein, the term “mutation” or “variant” refers to a protein comprising an alteration in the wild type, such as but not limited to, one or more amino acid deletions, insertions, and/or substitutions. A mutation in a protein may result in either inactivation, a decrease in activity or effect (e.g., binding), or an increase in activity or effect (e.g., binding or selectivity), or another altered property or effect of the protein.

In at least certain embodiments, the present disclosure is directed to a variant Cas12 protein, comprising: a nuclease (NUC) lobe, a recognition (REC) lobe, and a modified bridge helix (BH) region joining the NUC lobe and the REC lobe, the variant Cas12 protein having increased DNA cleavage selectivity relative to a corresponding wild type Cas12 protein, wherein the modified BH region increases the DNA cleavage selectivity of the variant Cas12 protein relative to the DNA cleavage selectivity of the corresponding wild type Cas12 protein, and wherein the modified BH region comprises at least one amino acid substitution relative to the corresponding wild type Cas12 protein. The at least one amino acid substitution in the modified BH region may be selected from the group consisting of ala, arg, asn, asp, cys, glu, gln, gly, his, ile, leu, lys, met, phe, pro, ser, thr, trp, tyr, and val. The at least one amino acid substitution in the modified BH region may comprise a substitution in any one or more of amino acid positions 956, 957, 961, 962, 969, and 970, wherein said amino acid positions are numbered relative to a Francisella tularensis novicida Cas12a (FnCas12a) protein set forth in of the amino acid sequence SEQ ID NO:1, and wherein the variant Cas12a protein has at least 90% identity to SEQ ID NO:1. The variant of SEQ ID NO: 1 may comprise at least a pair of substitutions selected from the group consisting of (a) substitutions in amino acid positions 956 and 957, (b) substitutions in amino acid positions 961 and 962, and (c) substitutions in amino acid positions 969 and 970. The at least one amino acid substitution in the modified BH region comprises a substitution in any one or more of amino acid positions 875, 876, 880, 881, 888, and 889, wherein said amino acid positions are numbered relative to a Lachnospiraceae bacterium Cas12a (LbCas12a) protein set forth in of the amino acid sequence SEQ ID NO:2, and wherein the variant Cas12a protein has at least 90% identity to SEQ ID NO: 2. The variant of SEQ ID NO:2 may comprise at least a pair of substitutions selected from the group consisting of (a) substitutions in amino acid positions 875 and 876, (b) substitutions in amino acid positions 880 and 881, and (c) substitutions in amino acid positions 888 and 889. The at least one amino acid substitution in the modified BH region comprises a substitution in any one or more of amino acid positions 943, 944, 948, 949, 956, and 957, wherein said amino acid positions are numbered relative to a Acidaminococcus sp strain BV3L6 Cas12a (AsCas12a) protein set forth in of the amino acid sequence SEQ ID NO:3, and wherein the variant Cas12a protein has at least 90% identity to SEQ ID NO:3. The variant of SEQ ID NO:3 may comprise at least a pair of substitutions selected from the group consisting of (a) substitutions in amino acid positions 943 and 944, (b) substitutions in amino acid positions 948 and 949, and (c) substitutions in amino acid positions 956 and 957. The at least one amino acid substitution in the modified BH region comprises a substitution in any one or more of amino acid positions 631, 632, 644, 645, 654, 655, and 656, wherein said amino acid positions are numbered relative to a Alicyclobacillus acidoterrestris strain ATCC 49025 Cas12b (AaCas12b) protein set forth in of the amino acid sequence SEQ ID NO:4, and wherein the variant Cas12b protein has at least 90% identity to SEQ ID NO: 4. The variant of SEQ ID NO:4 may comprise at least a pair of substitutions selected from the group consisting of (a) substitutions in amino acid positions 631 and 632, (b) substitutions in amino acid positions 644 and 645, (c) substitutions in amino acid positions 654 and 655, and (d) substitutions in amino acid positions 655 and 656. In other embodiments, the present disclosure is directed to a nucleic acid which encodes any of the variants described herein. The nucleic acid may be operably linked to a transcription control sequence. The nucleic acid may be disposed in an expression vector. In other embodiments, the present disclosure is directed to a cell comprising the expression vector. The cell may further comprise a nucleic acid comprising or encoding a guide RNA that directs the variant Cas12 protein to a target genomic sequence. In other embodiments, the present disclosure is directed to system comprising the variant Cas12 protein, and a Cas12 guide RNA. In other embodiments, the present disclosure is directed to method of gene editing, comprising using a variant Cas12 protein as described elsewhere herein in a CRISPR-Cas gene-editing procedure. For example, the variant Cas12 protein may be selected from the group consisting of: (a) a variant wherein the at least one amino acid substitution in the modified BH region comprises a substitution in any one or more of amino acid positions 956, 957, 961, 962, 969, and 970, wherein said amino acid positions are numbered relative to a Francisella tularensis novicida Cas12a (FnCas12a) protein set forth in of the amino acid sequence SEQ ID NO:1, and wherein the variant Cas12a protein has at least 90% identity to SEQ ID NO: 1; (b) a variant wherein the at least one amino acid substitution in the modified BH region comprises a substitution in any one or more of amino acid positions 875, 876, 880, 881, 888, and 889, wherein said amino acid positions are numbered relative to a Lachnospiraceae bacterium Cas12a (LbCas12a) protein set forth in of the amino acid sequence SEQ ID NO:2, and wherein the variant Cas12a protein has at least 90% identity to SEQ ID NO:2; (c) a variant wherein the at least one amino acid substitution in the modified BH region comprises a substitution in any one or more of amino acid positions 943, 944, 948, 949, 956, and 957, wherein said amino acid positions are numbered relative to a Acidaminococcus sp strain BV3L6 Cas12a (AsCas12a) protein set forth in of the amino acid sequence SEQ ID NO:3, and wherein the variant Cas12a protein has at least 90% identity to SEQ ID NO: 3; and (d) a variant wherein the at least one amino acid substitution in the modified BH region comprises a substitution in any one or more of amino acid positions 631, 632, 644, 645, 654, 655, and 656, wherein said amino acid positions are numbered relative to a Alicyclobacillus acidoterrestris strain ATCC 49025 Cas12b (AaCas12b) protein set forth in of the amino acid sequence SEQ ID NO:4, and wherein the variant Cas12b protein has at least 90% identity to SEQ ID NO: 4.

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components and compositions described herein, the methods described herein or in the steps or the sequence of steps of the methods described herein without departing from the spirit and scope of the present disclosure.

What is claimed is:

1. A variant Cas12 protein, comprising: a nuclease (NUC) lobe, a recognition (REC) lobe, and a modified bridge helix (BH) region joining the NUC lobe and the REC lobe, the variant Cas12 protein having increased DNA cleavage selectivity relative to a corresponding wild type Cas12 protein, wherein the modified BH region increases the DNA cleavage selectivity of the variant Cas12 protein relative to the DNA cleavage selectivity of the corresponding wild type Cas12 protein, and wherein the modified BH region comprises at least one amino acid substitution relative to the corresponding wild type Cas12 protein, and wherein the at least one amino acid substitution in the modified BH region comprises a substitution in any one or more of amino acid positions 631, 632, 644, 645, 654, 655, and 656, wherein said amino acid positions are numbered relative to a Alicyclobacillus acidoterrestris strain ATCC 49025 Cas12b (AaCas12b) protein set forth in of the amino acid sequence SEQ ID NO:4, and wherein the variant Cas12b protein has at least 90% identity to SEQ ID NO:4.

2. The variant Cas12 protein of claim 1, wherein the at least one amino acid substitution in the modified BH region is selected from the group consisting of ala, arg, asn, asp, cys, glu, gln, gly, his, ile, leu, lys, met, phe, pro, ser, thr, trp, tyr, and val.

3. The variant Cas12 protein of claim 1, wherein the at least one amino acid substitution in the modified BH region is selected from the group consisting of (1) Leu 631 and/or Arg 632 mutated to Pro 631 and/or Pro 632, (2) Gln 644 and/or Leu 645 mutated to Pro 644 and/or Pro 645, (3) Leu 654 and/or Leu 655 mutated to Pro 654 and/or Pro 655, and (4) Leu 655 and/or Val 656 mutated to Pro 655 and/or Pro 656.

4. The variant Cas12 protein of claim 1, comprising at least one pair of substitutions selected from the group consisting of (a) substitutions in amino acid positions 631 and 632, (b) substitutions in amino acid positions 644 and 645, (c) substitutions in amino acid positions 654 and 655, and (d) substitutions in amino acid positions 655 and 656.

5. The variant Cas12 protein of claim 4, wherein each substitution of the at least one pair of substitutions is independently selected from the group consisting of ala, arg, asn, asp, cys, glu, gln, gly, his, ile, leu, lys, met, phe, pro, ser, thr, trp, tyr, and val.

6. The variant Cas12 protein of claim 1, comprising substitutions in at least position 631 and position 632.

7. The variant Cas12 protein of claim 6, wherein the substitutions in position 631 and position 632 are independently selected from the group consisting of ala, arg, asn, asp, cys, glu, gln, gly, his, ile, leu, lys, met, phe, pro, ser, thr, trp, tyr, and val.

8. The variant Cas12 protein of claim 1, comprising substitutions in at least position 644 and position 645.

9. The variant Cas12 protein of claim 8, wherein the substitutions in position 644 and position 645 are independently selected from the group consisting of ala, arg, asn, asp, cys, glu, gln, gly, his, ile, leu, lys, met, phe, pro, ser, thr, trp, tyr, and val.

10. The variant Cas12 protein of claim 1, comprising substitutions in at least position 654 and position 655.

11. The variant Cas12 protein of claim 10, wherein the substitutions in position 654 and position 655 are independently selected from the group consisting of ala, arg, asn, asp, cys, glu, gln, gly, his, ile, leu, lys, met, phe, pro, ser, thr, trp, tyr, and val.

12. The variant Cas12 protein of claim 1, comprising substitutions in at least position 655 and position 656.

13. The variant Cas12 protein of claim 12, wherein the substitutions in position 655 and position 656 are independently selected from the group consisting of ala, arg, asn, asp, cys, glu, gln, gly, his, ile, leu, lys, met, phe, pro, ser, thr, trp, tyr, and val.

14. A method of gene editing, comprising using a variant Cas12 protein in a CRISPR-Cas gene-editing procedure, wherein the variant Cas12 protein comprises a nuclease (NUC) lobe, a recognition (REC) lobe, and a modified bridge helix (BH) region joining the NUC lobe and the REC lobe, the variant Cas12 protein having increased DNA cleavage selectivity relative to a corresponding wild type Cas12 protein, wherein the modified BH region increases the DNA cleavage selectivity of the variant Cas12 protein relative to the DNA cleavage selectivity of the corresponding wild type Cas12 protein, and wherein the modified BH region comprises at least one amino acid substitution relative to the corresponding wild type Cas12 protein, and wherein the variant Cas12 protein is a variant Cas12b protein wherein the at least one amino acid substitution in the modified BH region comprises a substitution in any one or more of amino acid positions 631, 632, 644, 645, 654, 655, and 656, wherein said amino acid positions are numbered relative to a Alicyclobacillus acidoterrestris strain ATCC 49025 Cas12b (AaCas12b) protein set forth in of the amino acid sequence SEQ ID NO:4, and wherein the variant Cas12b protein has at least 90% identity to SEQ ID NO:4.


SEQUENCE LISTING (4 sequences)

SEQ ID NO: 1 - protein, 1300 aa, Francisella tularensis
  source 1..1300: note = subsp. novicida (strain U112)
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 2 - protein, 1228 aa, unidentified
  source 1..1228: note = Lachnospiraceae bacterium ND2006
AASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 3 - protein, 1307 aa, Acidaminococcus sp.
  source 1..1307: note = (strain BV3L6)
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 4 - protein, 1129 aa, Alicyclobacillus acidoterrestris
  source 1..1129: note = (strain 49025)
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI